(19) United States
(12) Patent Application Publication
(10) Pub. No.: US 2025/0100951 A1
(43) Pub. Date: Mar. 27, 2025

(54) SELECTIVE AND LONG-LIVED SINGLE COMPONENT HETEROGENEOUS ETHYLENE TRIMERIZATION CATALYSTS ENABLED BY SURFACE LITHIATION

(71) Applicant: UChicago Argonne, LLC, Chicago, IL (US)

(72) Inventors: David Kaphan, Oak Park, IL (US); Massimiliano Delferro, Chicago, IL (US); Uddhav Ajay Kanbur, Naperville, IL (US); Cong Liu, Darien, IL (US)

(73) Assignee: UChicago Argonne, LLC, Chicago, IL (US)

(21) Appl. No.: 18/372,509

(22) Filed: Sep. 25, 2023

(51) Int. Cl. C07C 2/24; B01J 23/04; B01J 31/16; B01J 31/22

(52) U.S. Cl. CPC C07C 2/24; B01J 23/04; B01J 31/1608; B01J 31/2208; B01J 2231/20; B01J 2531/62; B01J 2531/72; C07C 2521/06; C07C 2523/04

(57) ABSTRACT

A catalyst for selective hexane and/or butene formation from ethylene. The catalyst is formed by grafting Cr or Mn to an inorganic support. The grafted metal site is reduced, forming a catalyst that is selective for selective trimerization of ethylene to hexene and/or butene.

### STATEMENT OF GOVERNMENT INTEREST

This invention was made with government support under Contract No. DE-AC02-06CH11357 awarded by the United States Department of Energy to UChicago Argonne, LLC, operator of Argonne National Laboratory. The government has certain rights in the invention.

### TECHNICAL FIELD

The present disclosure relates generally to selective and long-lived single component heterogeneous ethylene trimerization catalysts enabled by surface lithiation.

### BACKGROUND

The global linear α-olefin market is worth $9.5 Bn and is expected to grow to $15 Bn in the next ten years. These products have a diverse industrial application such as for the manufacture of plasticizers, fine chemicals, lubricants, detergent alcohols, polyethylene such as high density polyethylene (“HDPE”) and linear low density polyethylene (“LLDPE”). However, commercially viable manufacturing for such products lacks a catalytic material for selective, low energy, production of the desired α-olefin.

While a number of α-olefin production processes are desired, the development of heterogeneous catalysts for ethylene oligomerization is one of particular focus. However, ethylene oligomerization operating via the oxidative cyclization mechanism has presented challenges. Several prominent homogeneous systems for ethylene involving metallacycles have been developed and few have been commercialized. For example, Alphabutol process (butene) and Phillips trimerization systems (hexene) have been considered as benchmark standards for ethylene production. Yet, these systems are mainly comprised of a redox-active transition metal complex (e.g. chromium and titanium) and an alkylating agent (organoaluminum compounds). While such known processes may provide a pathway to ethylene, these present a number of significant disadvantages. Most commercial processes produce a mixture of linear α-olefins that need subsequent separation and purification. For example, typically, the activation of the aforementioned complexes leads to the formation of lower valent species capable of coordinating and oxidatively adding two ethylene molecules to form a metallacyclopentane.

A need remains in the art for a heterogeneous catalyst that is: (i) highly selective to oligomer formation with minimal polymerization, (ii) operate via the metallacycle mechanism, (iii) have high catalytic activities and (iv) have good catalyst longevity/recyclability. State-of-the-art catalysts do not meet all these requirements for a robust optimum catalyst.

### SUMMARY

In one embodiment, a method, comprising interacting a metal precursor having Cr or Mn with an inorganic support. A metal site is comprising Cr or Mn is grafted on the inorganic support. The metal site is reduced by exposure to a metal reductant. A reduced metal site is formed on the inorganic support, the reduced metal site being catalytically active in oxidative cyclization of ethylene via metallacycle intermediates.

In one embodiment, a catalyst having a support comprising lithium titanate, SiO₂, MgO, Al₂O₃, and carbon and a reduced metal site comprising a transition metal selected from the group consisting of Cr, Mn, Ti, V, Fe, Co, Ni, Ta, and W grafted to the support.

In one embodiment, a method of ethylene trimerization comprising interacting ethylene with a catalyst comprising a reduced metal site on an inorganic support and oligomerizing the ethylene, forming a mixture of hexene and butene.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF DRAWINGS

The foregoing and other features of the present disclosure will become more fully apparent from the following description and appended claims, taken in conjunction with the accompanying drawings. Understanding that these drawings depict only several implementations in accordance with the disclosure and are, therefore, not to be considered limiting of its scope, the disclosure will be described with additional specificity and detail through use of the accompanying drawings.

FIG. 1 illustrates a reaction scheme for the grafting of the metal site onto the support.

FIG. 2 illustrates a reaction scheme for the reduction scheme of the metal site.

FIG. 3 illustrates a scheme for the catalytic pathway.

FIG. 4 is a flow chart of a process for forming a catalyst in accordance with one embodiment.

FIG. 5 is a chart of experimental results.

FIG. 6 illustrates pressure profiles for the experimental results show in FIG. 5.

FIGS. 7A-B is a chart of experimental results.

FIG. 8A illustrates pressure profiles for the experimental results show in FIG. 5; FIG. 8B illustrates the experimental results in tabular form.

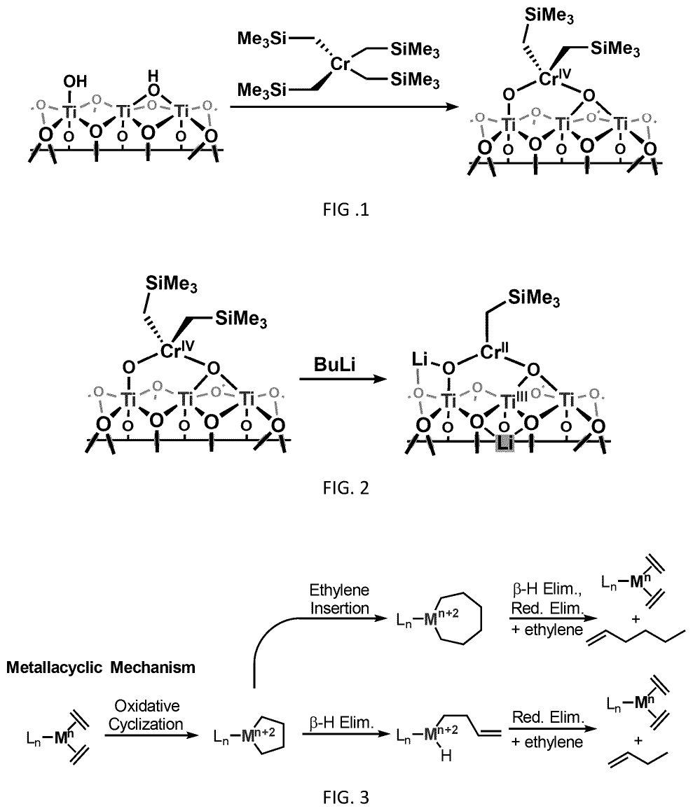

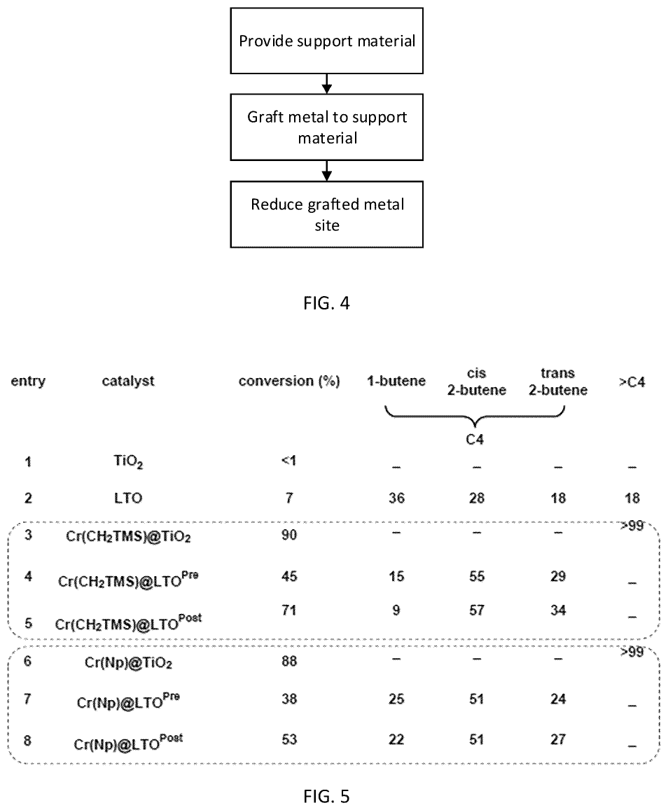

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION OF VARIOUS EMBODIMENTS

Before turning to the figures, which illustrate certain exemplary embodiments in detail, it should be understood that the present disclosure is not limited to the details of methodology set forth in the description or illustrated in the figures. It should also be understood that the terminology used herein is for the purpose of description only and should not be regarded as limiting.

Described herein is a catalyst and catalytic process that is (i) highly selective to oligomer formation with minimal polymerization, (ii) operate via the metallacycle mechanism, (iii) have high catalytic activities and (iv) have good catalyst longevity/recyclability. The present catalyst and processes address the lack of commercial, heterogeneous catalysts for selective ethylene trimerization. This provides an advantage over existing commercial catalysts, most of which are homogeneous and lead to a complex mixture of products that must be separated for different applications.

In one embodiment, a catalyst for selective hexene formation from ethylene. In one embodiment, Cr or Mn on LiₓTiO₂ is prepared by grafting the respective metal by reduction of the metal site with a reductant, such as butyllithium (BuLi). This process results in an active and selective catalyst for selective trimerization of ethylene to hexenes. In one embodiment, a Cr@LTO catalyst favors hexenes over butenes in an 80:20 ratio, while Mn@LTO catalyst provides C6/C4 in a ratio of 72:28.

The catalyst includes a catalytic metal that is grafted to or on a support. The catalytic metal may be selected from a variety of transition metals are amenable to grafting and should be able to facilitate ethylene trimerization, in particular Ti, V, Cr, Mn, Fe, Co, Ni, Cu, Zr, Nb, Mo, Ru, Rh, Pd, Ag, Hf, Ta, W, Re, Os, Ir, Pt and Au. In further embodiments, the catalytic metal is selected from Cr, Mn, Ti, V, Fe, Co, Ni, Ta, and W. In particular embodiments, the catalytic metal may be Cr or Mn. In one embodiment, the catalytic metal is a metal that is capable of participating in an oxidative cyclization for the conversion of ethylene to hexene and/or butene by way of metallacycle intermediates of the respective catalytic metal. Embodiments of a manganese-based ethylene trimerization catalyst and process described herein also provide the benefit of an ethylene production process that is less toxic than the present chromium-based systems. As further described below, embodiments provide a heterogeneous, potentially recyclable catalyst with good catalytic activity and excellent selectivity.

The catalyst includes a support to which, or on which, the metal is grafted. The catalyst support may be an inorganic catalyst support. For example, a metal oxide support such lithium titanates, including LiₓTiO₂ and lithium titanium oxide (LTO), SiO₂, MgO, and Al₂O₃, or a graphene/carbon. The form, such as nanoparticle, platelet, or the like, of the support will affect the lithium intercalation but it is not believed to alter the overall reduction of the metal center to a lower valent species. Notably, lithium intercalation in the surface is not believed to be important to promote this reaction. Further embodiments may utilize LTO-like materials, such as cathode materials such as Mn₂O₄ (LMO), LiCoO₂, LiFePO₄, LiNiO₂, which can offer electron rich surfaces that can stabilize a lower valent transition metal, such as Cr (II) or Mn (II) in particular embodiments. In one embodiment, the grafting is accomplished directly by a reductive grafting step involving the immobilization of a chromium complex onto lithium intercalated titanium oxide. In a further embodiment, further, reduced chromium catalyst can be obtained by lithiation of a titania-supported chromium complex. It is believed that the crystalline TiO₂ surface is important in the mechanism preventing the minority sites from forming, thus, in some embodiments ZrO₂ or HfO₂, may be utilized.

The formation of the catalyst utilizes a metal precursor in the grafting of the catalytic metal to the support. In some embodiments, grafting utilizes a homoleptic organometallic precursor. The reaction conditions for grafting may include a temperature of −30° C. to room temperature utilizing solvents such as pentane, hexane, DCM, ether, THF, benzene, and toluene. In one embodiment forms of Trimethylsilyl)methyllithium are utilized, such as a, Cr(CH₂TMS)₄ or [Mn(CH₂SiMe₃)₂]ₙ.

Once the metal is grafted to the support, the metal site is reduced by a metal reductant. The metal reductant is, in one embodiment, a metal alkyl reductant. The metal reductant should be should be capable of reducing the catalytic metal site. For reduction of the metal site, temperatures range from −30 C to room temperature, in some embodiments as low as −78° C., in solvents like THF, ether, pentane, hexane, benzene, cyclohexane, heptane, toluene. It is believed that an extended alkyl chain is required to perform the reduction, and so methyllithium, phenyllithium, or similar reagents are not expected to produce an active catalyst. Various alkyllithiums are expected to be effective in the reduction and intercalation step, especially ones with alkyl groups containing β-hydrogens such as nBuLi, sec-BuLi, tert-BuLi, as well as (BuNa), butylmagnesium (Bu₂Mg), butyltin (Bu₄Sn). LiCH₂CMe₃, LiCH₂SiMe₃, ethyllithium, isopropyllithium, and hexyllithium. Additionally, trialkyl aluminums AlR₃ and dialkylzincs ZnR₂ would also be capable of reducing the metal center. In particularly embodiments, including butyllithium (BuLi), butylsodium (BuNa), butylmagnesium (Bu₂Mg), or even possibly butyltin (Bu₄Sn). It is believed that the presence of a C—H bond in the beta position allows for inner sphere reduction of the metal by alkylation, beta-hydride elimination, and reductive elimination. This is, in particular, applicable for the Cr embodiments.

Embodiments of Cr@LTO in accordance with the discussion above were formed by formation of Cr. In one embodiment, the process for grafting the Cr to LTO (Cr@LTO^{Pre}) comprises:

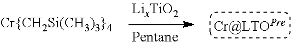

In a further embodiment, the grafted Cr @TiO₂ is reduced (Cr@LTO) by:

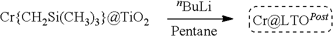

Surprisingly, the present embodiments do not result in either high molecular weight polyethylene (as in the Phillips catalyst) or produce statistical mixtures of olefins via an unselective, linear insertion mechanism (mechanistically distinct from the oxidative cyclization mechanism). In one embodiment, the ethylene trimerization by catalysts described herein results in a mixture of hexene and butene, substantially free of polymeric products with a carbon backbone greater than C6. Further, the combination of Cr and LTO alone is insufficient to achieve the described catalytic activity. Rather, it is the reaction of the Cr (or Mn) site with the metal reductant that produces the active site and the surprising catalytic activity and selectivity. When a grafted Cr site is reduced with Li Naphthalenide a different Cr/LTO material results that has no catalytic activity. While various supports may be utilized, LTO has been observed to provide surprising selectivity. For example, grafting the same Cr complex on SiO₂ followed by reduction with BuLi affords a spectroscopically similar Cr site, but polymerization occurs under catalytic conditions, exhibiting a mixture of hexenes and polyethylene.

In one embodiment, the resultant catalyst provides an activator-free system by leveraging the electronic properties of traditional inorganic supports. In this one embodiment, chromium on lithium titanium oxide (Cr@LTO) mediates the exclusive formation of butenes and hexenes, sustained over long reaction times. A comparable chromium active site can be generated by organometallic grafting on silica and subsequent reduction, however, in the absence of the electron rich LTO surface the silica supported material deactivates rapidly, underscoring the importance of the novel redox active surface approach to generate ethylene trimerization catalysts with extended lifetimes.

### Examples

TABLE 1
Ethylene oligomerization with Cr complexes.

| | | Ethylene | | | | | |
|---|---|---|---|---|---|---|---|
| | | Pressure | | | Productivity | Activity | # Total |
| Entry | Catalyst | (psi) | C₄% | C₆% | (g/g Cr/h) | (g/g Cr/h) | Turnovers |
| 1 | 1^{TiO2} | 15 | — | — | 12 | 0.7 | 22 |
| 2 | 1^{SiO2} | 15 | — | — | 4 | 0.3 | 9 |
| 3 | 1^{LTO} | 15 | 17 | 83 | 10 | 0.6 | 8 |
| 4 | 1^{Li/SiO2} | 15 | 23 | 77 | 9 | 0.5 | 6 |
| 5 | 1^{TiO2} | 100 | — | — | 2851 | 168 | 5358 |
| 6 | 1^{SiO2} | 100 | — | — | 1345 | 79 | 2505 |
| 7 | 1^{Li/SiO2} | 100 | 48 | 52 | 892 | 52 | 632 |
| 8 | 1^{LTO} | 100 | 20 | 80 | 464 | 27 | 309 |
| 12 | 1^{LTO} | 100 | | | | | |
| 13 | 1^{LTO-Inv} | 100 | 33 | 67 | 137 | 8 | 96 |
| 14 | 1^{LTO-LiNp} | 100 | | | | | |

As illustrated in Table 1, ethylene oligomerization by various samples varies in resultant product, productivity, and reactivity. For embodiments with a reduced metal site on LTO or Li/SiO₂ the, the products can be seen to be C4/C6 in contrast to existing catalytic process examples. Generally, the product distribution, even between hexene and butene, is likely governed by the stability of the chromium metallacycle formed on the electron rich surface, which are both steric and electronic effects. In Table 1, 1LTO-Inv is prepared by the reaction of Cr(CH2SiMe3)4 with LTO (lithium titanium oxide) in pentane at room temperature. The elemental composition of 1LTO-Inv is (wt %): Ti (43.07%), Cr (0.97%) and Li (5.06%).

FIG. 5 illustrates a comparison of several control sample as well as embodiments described herein. Entries 1 and 2 are both bare substrates. While LTO results in some butene, it also includes a range of oligomers. Entries 3-5 are Cr(CH₂TMS)₄ on the noted substrates, titanium dioxide (entry 3) as well as LTO both pre and post reduction. Entries 6-8 are Cr(NP)LTO, where “NP” is neopenyl, reflecting a minor variant of the standard metal precursor having in the ligand of the precursor, the silicon of the TMS replaced with a carbon to make the neopentyl.

The examples utilize LTO (bare) formed as shown in Figure

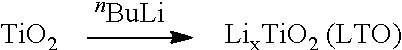

The examples utilize Cr grafted on TiO₂ formed by:

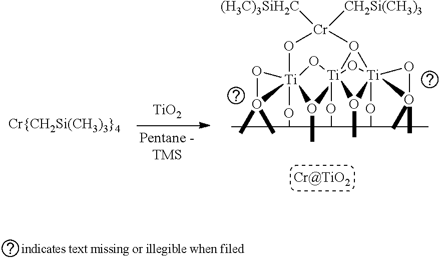

The examples utilize Cr grafted on SiO₂ formed by:

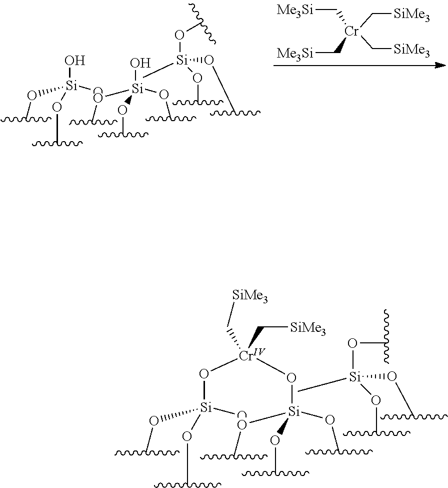

Further, the examples utilize reduced Cr site on SiO₂ formed by:

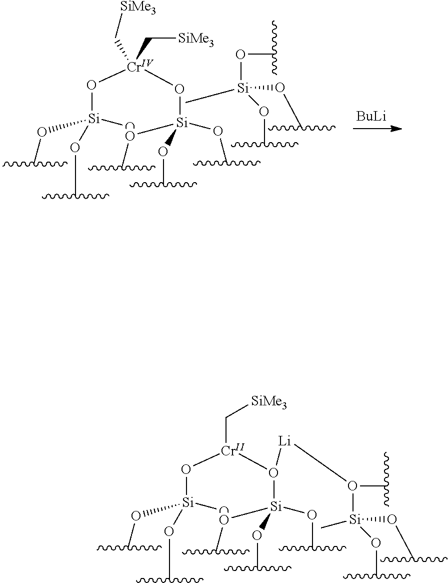

One set of experiments were performed in accordance with the reaction process below. The resultant products are shown generally, with the results illustrated in FIG. 5 with oligiomers grouped as >C4 category. The entries in accordance with embodiments described herein, 4-5 and 7-8 illustrate selectivity for butenes, with improved conversion rate for the entries that were reduced grafted materials.

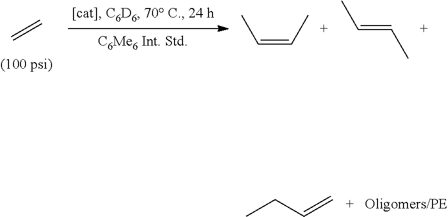

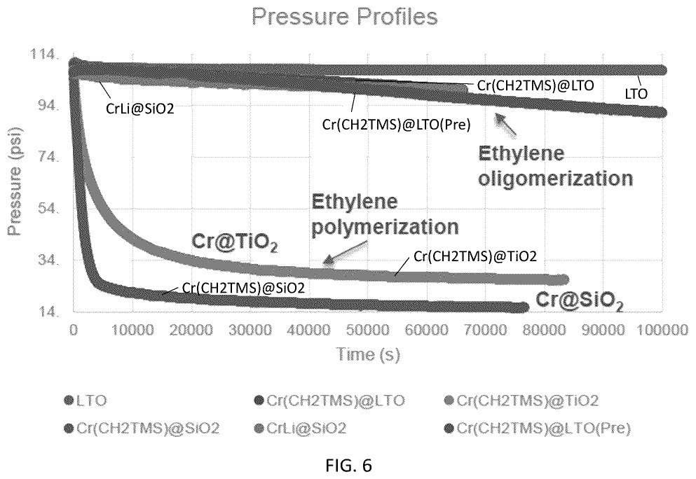

FIG. 6 illustrates the pressure profiles in the reactor under conditions noted above for the respective products. Ethylene oligomerization is characterized in the region noted. FIG. 6 illustrates the polymerization observed for the prior art entries in contrast for embodiments as described herein (as well as bare LTO) exhibiting oligomerization.

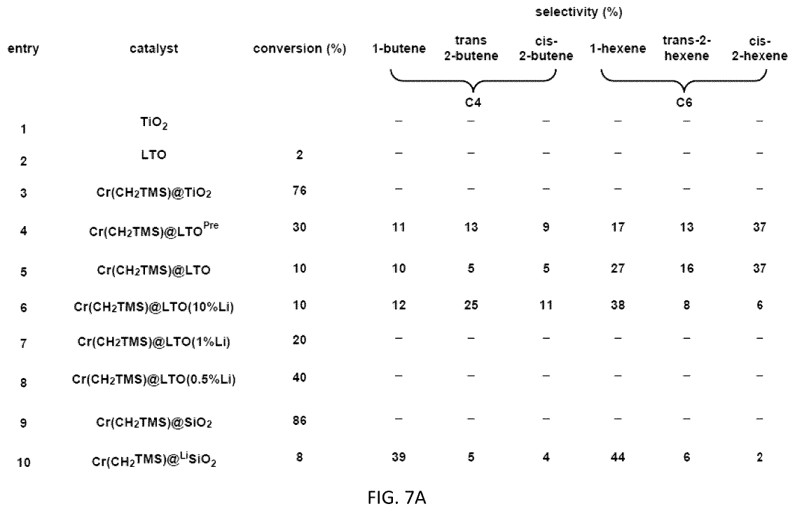

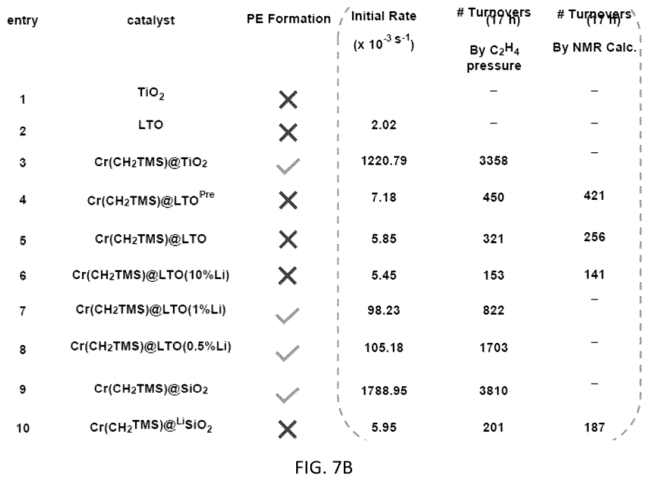

One set of experiments were performed in accordance with the reaction process below. The resultant products are shown generally, with the results illustrated in FIG. 7A-B with oligiomers grouped as >C4 category.

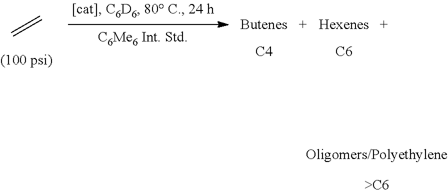

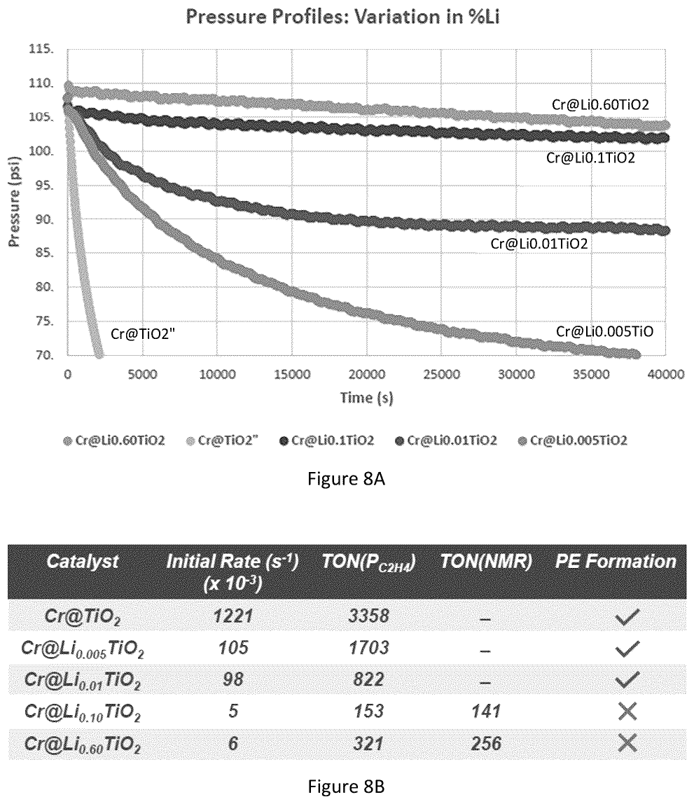

FIG. 8A illustrates the pressure profile for the recited examples under the reaction scheme above. FIG. 8B includes results for the entries of FIG. 8A, with lithiation impacting the oxidation state, which is observed to have a significant impact on the resultant products of the reaction. An oxidation state for Ti below is associated with oligomerization while an oxidation state of 4 is associated with polymerization.

### Definitions

No claim element herein is to be construed under the provisions of 35 U.S.C. § 112(f), unless the element is expressly recited using the phrase “means for.”

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic. For example, circuit A communicably “coupled” to circuit B may signify that the circuit A communicates directly with circuit B (i.e., no intermediary) or communicates indirectly with circuit B (e.g., through one or more intermediaries).

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

What is claimed is:

1. A method, comprising:
interacting a metal precursor having Cr or Mn with an inorganic support;
grafting a metal site comprising Cr or Mn on the inorganic support;
reducing the metal site by exposure to a metal reductant; and
forming a reduced metal site on the inorganic support, the reduced metal site being catalytically active in oxidative cyclization of ethylene via metallacycle intermediates

2. The method of claim 1, wherein the metal precursor is comprises trimethylsilyl)methyllithium.

3. The method of claim 2, wherein the metal precursor is selected from a group consisting of: Cr(CH₂TMS)₄ or Mn(CH₂TMS)₄.

4. The method of claim 1, wherein the metal reductant is selected from a group consisting of: butyllithium (BuLi), butylsodium (BuNa), butylmagnesium (Bu₂Mg), butyltin (Bu₄Sn), LiCH₂CMe₃, LiCH₂SiMe₃, ethyllithium, isopropyllithium, and hexyllithium.

5. The method of claim 4, wherein the metal reductant is butyllithium (BuLi).

6. The method of claim 1, wherein the support is selected from the group consisting of lithium titanate, SiO₂, MgO, Al₂O₃, and carbon.

7. The method of claim 6, wherein the support comprises LiₓTiO₂ or lithium titanium oxide (LTO).

8. The method of claim 1, wherein the support comprises a lithium titanate and further wherein the metal precursor is selected from a group consisting of: Cr(CH₂TMS)₄ or Mn(CH₂TMS)₄ and further wherein the metal reductant is selected from a group consisting of: butyllithium (BuLi), butylsodium (BuNa), butylmagnesium (Bu₂Mg), butyltin (Bu₄Sn), LiCH₂CMe₃, LiCH₂SiMe₃, ethyllithium, isopropyllithium, and hexyllithium.

9. A catalyst comprising:
a support comprising lithium titanate, SiO₂, MgO, Al₂O₃, or carbon; and
a reduced metal site comprising a transition metal selected from the group consisting of Cr, Mn, Ti, V, Fe, Co, Ni, Ta, and W grafted to the support.

10. The catalyst of claim 9, wherein the support comprises lithium titanate.

11. The catalyst of claim 10, wherein the support comprises LTO.

12. The catalyst of claim 10, wherein the reduced metal site comprises Cr or Mn.

13. The catalyst of claim 12, wherein the reduced metal site comprises Cr (II).

14. A method of ethylene trimerization comprising:
interacting ethylene with a catalyst comprising a reduced metal site on an inorganic support; and
oligomerizing the ethylene, forming a mixture of hexene and butene.

15. The method of claim 14, wherein, after interacting ethylene with the catalyst, undergoing metalacyclic process.

16. The method of claim 15, wherein the metalacyclic process comprises:
oxidative cyclization, beta-hydride elimination, and reductive elimination.

17. The method of claim 16, wherein, prior to beta-hydride elimination, performing ethylene insertion.

18. The method of claim 14, wherein the reduced metal site comprises Cr.

19. The method of claim 14, wherein the reduced metal site comprises Mn.
